(19) United States
(12) Patent Application Publication
(10) Pub. No.: US 2026/0220774 A1
(43) Pub. Date: Jul. 30, 2026

(54) ESTIMATING DIFFUSE PLAQUE BURDEN OF A VESSEL

(71) Applicant: KONINKLIJKE PHILIPS N.V., EINDHOVEN (NL)

(72) Inventors: Tobias WISSEL, Lübeck (DE); Christian HAASE, HAMBURG (DE); Michael GRASS, BUCHHOLZ IN DER NORDHEIDE (DE)

(21) Appl. No.: 19/148,526

(22) PCT Filed: Jan. 26, 2024
(86) PCT No.: PCT/EP2024/051885

§ 371 Date: Jul. 16, 2025

(30) Foreign Application Priority Data
Jan. 26, 2023 (EP) 23153386.0

(51) Int. Cl. G06T 7/00; G06T 5/70; G06T 7/11; G06T 7/62; G16H 50/30

(52) U.S. Cl. CPC G06T 7/0012; G06T 5/70; G06T 7/11; G06T 7/62; G16H 50/30; G06T 2207/10116; G06T 2207/20024; G06T 2207/20076; G06T 2207/20081; G06T 2207/20084; G06T 2207/20182; G06T 2207/30101

(57) ABSTRACT

There is provided a computer-implemented method of estimating a diffuse plaque burden of a vessel. The computer-implemented method comprises receiving S1 an angiogram 1000 including the vessel; identifying S2 a lumen of the vessel in the angiogram; approximating S3 a healthy lumen of the vessel based on determining a change in the lumen along a length of the vessel; and estimating S4 the diffuse plaque burden of the vessel based on subtracting the approximation of the healthy lumen from the lumen.

### FIELD OF THE INVENTION

The invention relates to a computer-implemented method of estimating a diffuse plaque burden of a vessel.

### BACKGROUND TO THE INVENTION

Vascular disease manifests itself in various forms. A particular risk of, for example, coronary artery disease (CAD) is that it may lead to myocardial infarction, ischemia and death.

A typical recommended diagnostic analysis in a stable patient is a pressure measurement over stenotic vessel segments including one or multiple localized lesions to categorize the impairment of blood flow through the vessel caused by the one or multiple localized lesions. The result of this diagnostic analysis is used to decide whether a stent should be placed or not. However, such analyses do not fully represent the overall disease burden of the vessel.

In particular, an important feature of a diseased vessel tree is an atypical rough vessel geometry. In a diseased state the vessels widen and narrow due to plaque accumulation, small injuries to the vessel wall as well as the resulting atypical wall shear stress and pressure distributions. Apart from manifesting in stenoses i.e. localized lesions deemed significant by established scoring system (e.g. lesions with a SYNTAX score above 50%), possibly lengthy diffuse lesions exhibiting mild or moderate vessel narrowing are not accounted for in current scoring systems. However, these may clearly indicate diseased vasculature as an important marker when it comes to judging the systemic extent and future progression of the disease, or when patency of a therapy result is considered.

Hence, there is a need for an improved method of analyzing the disease burden of a vessel. More specifically, there is a need for a method of analyzing the disease burden of a vessel that accounts for the diffuse disease burden.

### SUMMARY OF THE INVENTION

The subject-matter of the present disclosure provides a computer-implemented method which at least partially obviates or mitigates at least some of the disadvantages of the prior art, whether identified herein or elsewhere, or to provide an alternative approach. For instance, it is an aim of embodiments of the invention to provide a method of analyzing the disease burden of a vessel that accounts for the diffuse disease burden.

According to the present disclosure there is provided a computer-implemented method and a transitory or non-transitory computer-readable medium, as set forth in the appended claims. Other features of the invention will be apparent from the dependent claims and the description that follows.

According to a first aspect, there is provided a computer-implemented method of estimating a diffuse plaque burden of a vessel. The computer-implemented method comprises receiving an angiogram including the vessel; identifying a lumen of the vessel in the angiogram; applying a local lesion identifying model configured to identify local lesions in the vessel and distinguish a first segment of the vessel from a second segment of the vessel including the identified local lesions; approximating a healthy lumen of the vessel at least for the first segment based on determining a change in the lumen along a length of the vessel; and estimating the diffuse plaque burden of the vessel based on subtracting the approximation of the healthy lumen from the lumen for the first segment.

As a result of applying the local lesion identifying model, one or more vessel segment(s) i.e. the “second vessel segment” including local lesions with significant, localized vessel narrowing (e.g. stenoses with a SYNTAX score above 50%) can be distinguished from (an)other vessel segment(s) i.e. the “first vessel segment” containing no lesions or lengthy diffuse lesions exhibiting mild or moderate vessel narrowing.

It is envisaged herein to analyze the disease burden for a vessel that does not contain any significant, local lesions. In that case, the local lesion identifying model does not identify any local lesions and the entire portion of the vessel to be analyzed further for diffuse disease burden is marked as the first segment. However, more advantageously, an estimate can be obtained of the diffuse disease burden in a vessel that contains both local and diffuse lesions, whereby insight can be obtained in the disease burden that would remain after treating significant lesions in the vessel by stenting or ballooning for example.

Suitable distinction between first and second vessel segments may be readily provided by local lesion identifying models as available in the state of the art. Nevertheless, in certain examples, a SYNTAX score may additionally be inputted by a user for fine-tuning a distinction between the first and second vessel segments if so required.

The angiogram may comprise a plurality of pixels. Identifying the lumen of the vessel in the angiogram may comprise identifying pixels of the plurality of pixels which define the lumen. In particular, the fine-scale resolution of a 2D X-ray (projection) angiogram is suited for estimating the diffuse disease burden as local plaque variations on higher spatial frequency levels are maintained, whereas such variations may be smoothened out in other imaging modalities.

The method may further comprise generating a spatial domain signal of dimensional variations along the length of the vessel. That is, a spatial domain signal is generated that is representative of the lumen dimension, such as diameter or alternatively cross-section, along the length of the vessel.

In certain examples, for the second segment, the spatial domain signal is set to a constant value e.g. zero, so that local lesions visible in the angiogram are removed from the analysis.

After distinguishing the first and second vessel segments, the healthy lumen is approximated at least for the first segment. It is known that healthy vessels are widest at their ostium and smoothly decrease in size with a more distal location. Therefore, an approximation of a healthy lumen is made based on this knowledge, from which, for the first segment, the spatial domain signal representing the actual lumen profile is then subtracted to obtain an indication of disease related widening and narrowing on a diffuse scale. In turn, this allows a numerical integration of the diffuse disease burden for a specific vessel or the full vascular tree, focusing only on the vessel portions outside significant localized lesions so as to, for example, determine the (diffuse) disease burden that remains once the local lesions will have been treated by stenting or ballooning.

Several examples for healthy vessel approximation are envisaged herein, which are set out further in the following.

In a first example, approximating the healthy lumen comprises extracting a low frequency content based on the spatial domain signal using a low-pass filter.

In a further example, approximating the healthy lumen may comprise applying the spatial domain signal to an isotonic regression model to fit a line of isotonic regression.

In a further example, approximating the healthy lumen may comprise applying the spatial domain signal to a component analysis, CA, model trained to determine an eigenvector that relates healthy lumens to lumens in which diffuse plaque has been introduced.

In a further example, approximating the healthy vessel may comprise applying the spatial domain signal to a neural network trained to determine a spatial domain signal of a healthy lumen of a vessel from a spatial domain signal of a lumen of a vessel.

The neural network may have been trained using spatial domain signals of paired images of healthy lumens and lumens in which diffuse plaque has been introduced.

Identifying the lumen of the vessel may comprise determining a center line of the lumen; mapping the center line onto a longitudinal axis; and correcting for any foreshortening of the lumen by resampling the lumen along the longitudinal axis.

In certain examples, for any second segment including local lesions, the centerline may be removed or disregarded. For example, in the above steps, a second segment may be removed from the lumen, such that a mapped and resampled representation of only the first segment(s) is obtained. Alternatively, any second segments are maintained in the mapped and resampled representation but their proximal and distal end points are marked therein, allowing the second segments to be easily disregarded during the further analysis.

In addition to distinguishing first and second segments, the method may further comprise identifying a branching point in the lumen; and assigning a portion of the vessel comprising the branching point to the second segment. In other words, vessel portions including branching points may thus be excluded from the further analysis, similar to what is being done for local lesions.

The method may further comprise applying a signal windowing function to the lumen to remove artifacts caused by sudden changes to lumen geometry.

While preferably relying on 2D X-ray (projection) angiograms for identifying the lumen, identifying the lumen may additionally or alternatively comprise identifying the lumen based on at least one of: a computer tomography, CT, scan and an intravascular ultrasound scan, IVUS.

Estimating the diffuse plaque burden of the vessel may be further based on: calculating a metric representative of the diffuse plaque burden of the vessel, the metric being at least one of: a spectral power; a variance of the estimated plaque burden for intervals along the vessel; a standard deviation of the estimated plaque burden for intervals along the vessel; an absolute value of the estimated plaque burden; and a proportion of the vessel having an estimated plaque burden above a predetermined value.

The method may further comprise outputting the estimation of the diffuse plaque burden to a user interface.

The change in the lumen may be a change in one of: a radius, a diameter and a cross-sectional area.

Receiving an angiogram may comprise receiving a plurality of angiograms. Identifying a lumen of the vessel in the angiogram may comprise identifying the lumen of the vessel in the plurality of angiograms.

The vessel may be a coronary vessel.

According to a second aspect, there is provided a transitory, or non-transitory, computer-readable medium having instructions stored thereon which, when executed by a processor, cause the processor to carry out the method of the first aspect.

These and other aspects of the present invention will be apparent from and elucidated with reference to the embodiment(s) described hereinafter.

### BRIEF DESCRIPTION OF THE DRAWINGS

For a better understanding of the invention, and to show how embodiments of the same may be brought into effect, reference will be made, by way of example only, to the accompanying diagrammatic Figs, in which:

FIGS. 1A and 1B each show a flowchart of a computer-implemented method of estimating a diffuse plaque burden of a vessel, according to an embodiment;

FIG. 2 shows an angiogram of a vessel having a diffuse plaque burden (Dinakar Bootla, Pruthvi C Revaiah, Navjyot Kaur, Yash Paul Sharma, Himanshu Gupta, The Use of Complementary Technologies in Calcified Left Main Disease: A Case Series, Journal of Asian Pacific Society of Cardiology 2022; 1:e13.);

FIGS. 3A and 3B respectively schematically depict a cross-section of a healthy lumen of a vessel and a cross-section of a lumen of the vessel in having a diffuse plaque burden, according to an embodiment;

FIG. 4 shows identification of contours on two angiograms and corresponding stretched lumen views;

FIG. 5 shows a graph depicting a change in a dimension of a lumen along a length of a vessel and an approximation of a change in the dimension of a healthy lumen of the vessel; and

FIGS. 6A and 6B respectively show perspective views of a vascular tree and the vascular tree in which a local lesion has been introduced.

### DETAILED DESCRIPTION

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

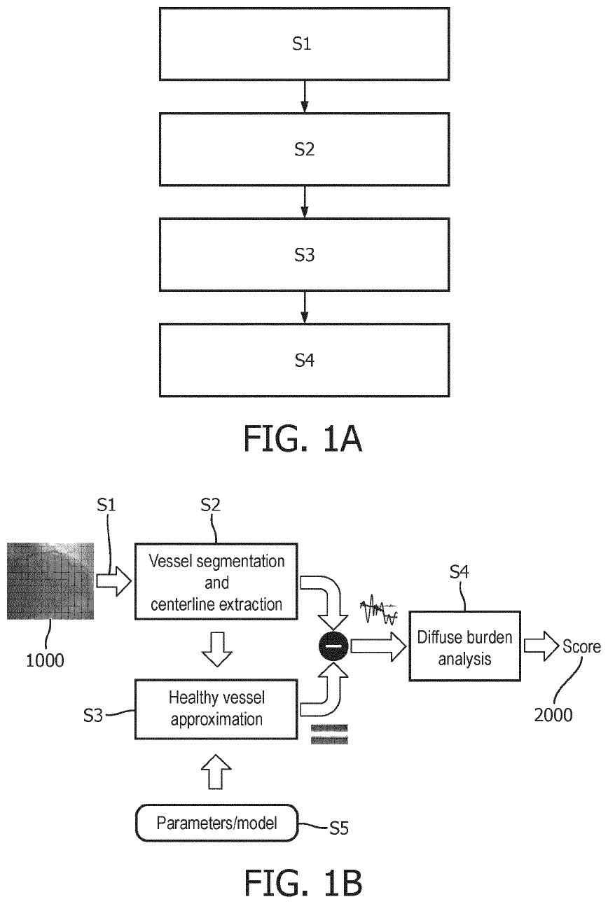

FIG. 1A shows a flowchart a computer-implemented method of estimating a diffuse plaque burden of a vessel (e.g., a coronary vessel), according to an embodiment. As shown in FIG. 1A, the method comprises receiving S1 an angiogram including the vessel; identifying S2 a lumen of the vessel in the angiogram; approximating S3 a healthy lumen of at least the first segment of the vessel based on determining a change in the lumen along a length of the vessel (e.g., a change in a radius, a diameter, a cross-sectional area or X-ray intensity along the vessel, which can serve as a proxy for vessel thickness in the projection direction); and estimating S4 the diffuse plaque burden of the vessel based on subtracting the approximation of the healthy lumen from the lumen for the first segment of the vessel

In this example, the identifying step S2 further includes the step of applying a local lesion identifying model configured to identify local lesions in the vessel and distinguishing a first segment of the vessel from a second segment of the vessel including the identified local lesions.

For example, the step of applying a local lesion identifying model may include identifying a local lesion from an angiogram using artificial intelligence (AI) techniques. For instance, a trained network is used that has been trained to mark lesions on the vessel tree visible in angiograms by learning local lesion characteristics from ground truth data, such as angiograms that have been manually annotated by users. The model may identify local lesions and then use this information to mark the corresponding vessel segments, in which the identified local lesions are located, as the second segment. Subsequently, the other portions of the vessel apart from the second segment are marked as the first segment to be used for further analysis of the diffuse disease burden.

FIG. 1B shows a flowchart of the same computer-implemented method. As described above in relation to FIG. 1A, the method comprises receiving S1 at least one angiogram 1000 including the vessel. As shown in FIG. 1B and as discussed further below, identifying S2 a lumen of the vessel in the angiogram may comprise determining a center line. As described above in relation to FIG. 1A, the method comprises approximating S3 a healthy lumen of the vessel. As indicated in FIG. 1B, approximating S3 a healthy lumen of the vessel includes performing S5 various parameterization and modelling, which is described in detail below. As shown in FIG. 1B, estimating S4 the diffuse plaque burden of the vessel is based on subtracting the approximation of the healthy lumen from the lumen. As indicated in FIG. 1B and as described in more detail below, the estimated diffuse plaque burden may be represented as a score or metric 2000.

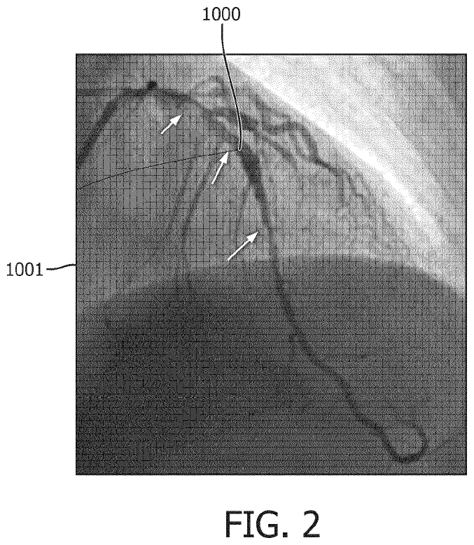

An angiogram is preferably a two-dimensional (2D) image, obtained by taking an X-ray of a vessel filled with contrast medium. An example of an angiogram 1000 is shown in FIG. 2. The angiogram 100 shown in FIG. 2 shows a vessel 1001 having a diffuse plaque burden. Advantageously, angiograms provide a fine-scale resolution that is particularly suited to estimating the diffuse burden, because plaque variation on a higher spatial frequency level is not smoothened out by the imaging process.

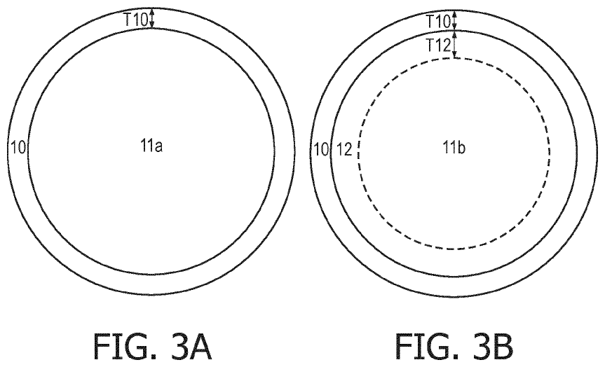

The term “lumen” is used in the conventional sense to refer to the interior space of a vessel through which blood typically flows. What is meant by the term “lumen,” and how the diffuse plaque burden of the vessel of FIG. 1A is estimated can be further understood with the aid of FIGS. 3A and 3B.

FIGS. 3A and 3B respectively schematically depict a cross-section of a healthy lumen 11a of a vessel and a cross-section of a lumen 11b of the vessel having a diffuse plaque burden, according to an embodiment. In FIG. 3A, the healthy lumen 11a is defined by the vessel wall 10, which has a thickness T10. In FIG. 3B, the lumen 11b is defined by the vessel wall 10, having the thickness T10, and plaque 12, having a thickness T12. Consequently, the thickness T12 of the plaque is equivalent to the difference between the dimensions (e.g., radius, diameter, cross-sectional area) of the healthy lumen 11a and the dimensions (e.g., radius, diameter, cross-sectional area) of the lumen 11b with a diffuse plaque burden. In other words, as underlined by comparing FIGS. 3A and 3B, by subtracting the dimensions of the lumen 11b from the healthy lumen 11a, the diffuse plaque burden can be determined. Therefore, if the healthy lumen of a diseased vessel (i.e., a vessel having a diffuse plaque burden) is approximated, the diffuse plaque burden of the vessel in question can be estimated through this subtraction.

Receiving S1 an angiogram 1000, as shown in FIG. 1B, may comprise receiving a plurality of angiograms 1000. Thus, identifying S2 the lumen of the vessel in the angiogram 1000 may comprise identifying the lumen of the vessel in the plurality of angiograms 1000. Advantageously, using a plurality of angiograms 1000 may facilitate more accurate estimation of the diffuse plaque burden by eliminating, for example, artifacts resulting from different cardiac activity. In case of 2D X-ray angiograms, the plurality of angiograms may include a plurality of angiograms acquired from a single projection direction or from multiple projection direction. The latter may, for example, facilitate the use of vessel cross-sections as a basis for the generated spatial domain signal.

The angiogram(s) 1000 may comprise a plurality of pixels such that identifying S2 the lumen of the vessel in the angiogram 1000 may comprise identifying pixels of the plurality of pixels which define the lumen.

Identifying S2 the lumen of the vessel may include identifying and segmenting the vessel(s) of interest from the angiogram image data. In an embodiment, a 2D angiographic X-ray frame is received as input, which shows the (coronary) vascular tree including the vessel(s) of interest, filled with contrast medium. The contrasted vessel tree is separated from the background using standard image processing, using a trained neural network or a combination thereof.

Further, a local lesion identifying model configured to identify local lesions in the vessel is applied, further segmenting the vessel(s) of interest by distinguishing a first segment of the vessel from a second segment of the vessel including the identified local lesions. The lumen of the second segment of the vessel may then be removed or disregarded for the further analysis.

For example, the step of applying a local lesion identifying model may include identifying a local lesion from an angiogram using artificial intelligence (AI) techniques. For instance, a trained network is used that has been trained to mark lesions on the vessel tree visible in angiograms by learning local lesion characteristics from ground truth data, such as angiograms that have been manually annotated by users. The model may identify local lesions and then use this information to mark the corresponding vessel segments, in which the identified local lesions are located, as the second segment. Subsequently, the other portions of the vessel apart from the second segment are marked as the first segment to be used for further analysis of the diffuse disease burden.

Identifying S2 the lumen of the vessel may further comprise determining a center line of the lumen. For example, in the segmented angiogram, those pixels defining the interface between the vessel wall and the lumen may be identified at least for the first segment. Subsequently, the center line of the lumen can be identified through the relative positions of the pixels corresponding to the interface between the vessel wall and the lumen. For instance, in the case of the lumen 11b shown in FIG. 3B, the center line of the lumen 11b is equidistant from each of the pixels identified as defining the interface between the plaque and the lumen 11b. In other words, the center line of the lumen 11b is the longitudinal axis of the cylinder having a cross-section corresponding to the dashed line defining the lumen 11b. Identifying the lumen of the vessel may be performed using standard image processing, a neural network or a combination thereof.

Subsequent to determining the center line of the lumen, identifying S2 the lumen may comprise mapping the center line onto a longitudinal axis. That is, the center line may be resampled/morphed into a stretched two-dimensional version in which the formerly sinuous centerline is mapped to a straight longitudinal axis (stretched lumen view). By stretching the lumen along a (straight) longitudinal axis, a better representation of the vessel is realized in the event of any foreshortening of the lumen that may be a result of an angle at which the X-ray was performed to generate the angiogram, for instance.

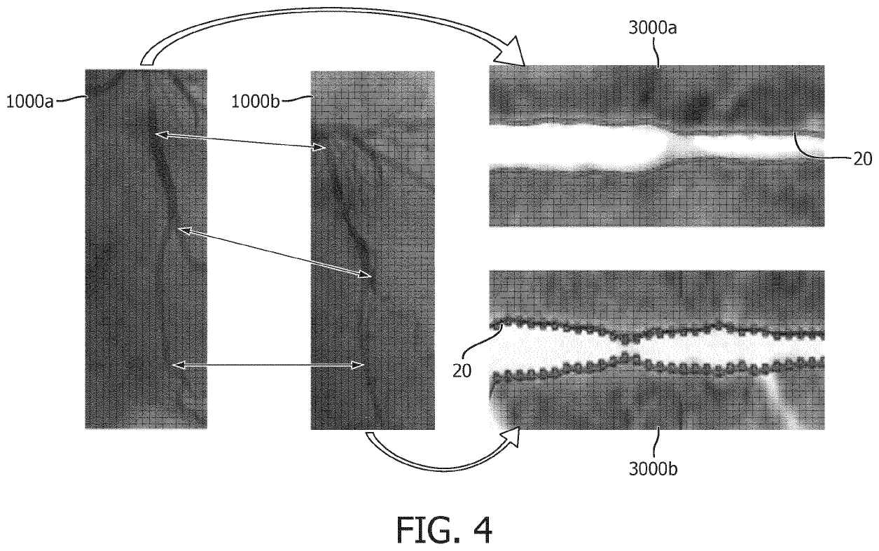

FIG. 4 shows center lines determined in two angiograms 1000a, 1000b. Adjacent to the angiograms 1000a, 1000b are shown corresponding stretched lumen views 3000a, 3000b of the vessels. Lines 20 in FIG. 4 indicate contours of the interface between the vessel wall and any plaque with the lumens.

Identifying S2 the lumen in the angiogram may additionally comprise identifying the lumen based on one or more of a computer tomography, CT, scan and an intravascular ultrasound scan, IVUS. By combining different inputs, advantageously, more accurate estimation of the diffuse plaque burden may be facilitated.

The method typically includes generating a spatial domain signal of dimensional variations along the length of the vessel. That is, a spatial domain signal is generated that is representative of the lumen dimension, such as diameter or alternatively cross-section, along the length of the vessel.

Following generating the spatial domain signal of dimensional variations along the length of the vessel, the healthy lumen can be approximated.

The, possibly reformatted, vessel representation of the first segment(s) and in particular the lumen contours contain two pieces of information: (1) a generally decaying trend in the vessel caliber or diameter in the direction from proximal to distal and (2) diffuse plaque burden. Optionally, the lumen contours for the second segment contain further information regarding (3) significant stenotic lesions.

Approximating S3 the healthy lumen includes, at least for the first segment, approximating the outline of a healthy vessel corresponding as closely as possible to the actual lumen outline. Preferably, in the healthy lumen approximation, the diffuse plaque burden in the vessel representation is removed as much as possible, as detailed in the four examples below.

In a first example, approximating S3 the healthy lumen comprises extracting a low frequency content based on the spatial domain signal using a low-pass filter. Preferably a 1D low-pass filter is used that extracts the low frequency information indicative of the decaying trend of the vessel caliber and filters out the higher frequency content that is representative of the diffuse plaque burden. In an example, the filter is applied separately to each vessel segment of at least the first vessel segment, to avoid discontinuity artefacts at boundaries between the first and second vessel segment. In certain examples, the same is done when the second vessel segment includes branching points, thereby similarly avoiding discontinuity artefacts at vessel branching points.

In certain embodiments, if the healthy lumen approximation includes both the first and second segments, an additional, distinct, low-pass filter may be used that is tuned to the unique signature of spatial frequencies of local lesions i.e. significant narrowings of the vessel having a relatively sparse distribution along the vessel as opposed to diffuse plaque.

In this first example, extracting the low frequency content may comprise converting the spatial domain signal to a frequency domain signal. Typically, the spatial domain signal is converted to the frequency domain signal by applying a Fourier transformation to the spatial domain signal. However, the low frequency content may also be extracted by applying the low-pass filter in the spatial domain directly, for example, in the case of a finite impulse response (FIR) filter or an infinite impulse response (IIR) filter. Use of a low-pass filter is, advantageously, a computationally efficient way by which to approximate the healthy lumen. The output from the filter is a frequency domain or time domain signal corresponding to only low frequency components of the input signal. The low frequency components correspond to an approximation of the healthy lumen. If a frequency domain signal is output, an inverse Fourier transform may be performed if a spatial domain signal is desired.

In a second example, approximating S3 the healthy lumen comprises applying the spatial domain signal to an isotonic regression model to fit a line of isotonic regression. Isotonic regression is also known as monotonic regression. As with linear regression models, isotonic regression models seek to minimize the mean-squared error. However, unlike linear regression models, isotonic regression models are freeform. In other words, isotonic regression models fit a piecewise-constant, monotonic function to data. In this second example an isotonic regression model is applied to the spatial domain signal, thereby fitting a line of isotonic regression to the spatial domain signal such that the change in the lumen along the vessel is determined, the change in the lumen along the vessel serving as an approximation of the healthy vessel. The line of isotonic regression is the output from the isotonic regression model. The line of isotonic regression is an approximation of a healthy lumen. Advantageously, as alluded to, isotonic regression is not constrained by a particular functional form. However, in this example, the isotonic regression model is preferrably applied to the first vessel segment only, requiring removal of the second vessel segment having (a) local lesion(s) in the previous step should these indeed be present.

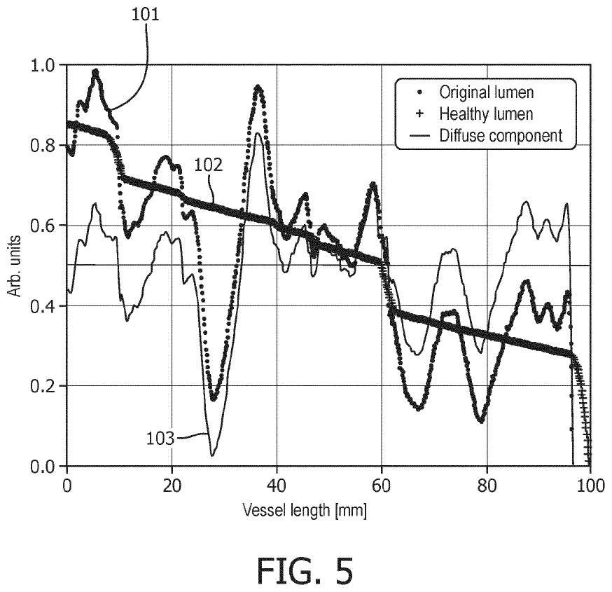

FIG. 5 shows a graph depicting a change in a dimension of a lumen of along a length of a vessel (line 101) and an approximation of a change in the dimension cross-sectional area of a healthy lumen of the vessel (line 102). The x-axis shows the length of the vessel in millimeters, and the y-axis shows signal intensity for the aforementioned dimensions (e.g., radius, diameter, cross-sectional area) in arbitrary units. In FIG. 5, the approximation of the cross-sectional area of the healthy lumen of the vessel has been determined by fitting an isotonic line of regression. The thin line 103 shows the difference between the observed lumen signal 101 and the approximated healthy lumen signal 102 by means of isotonic regression. The thin line 103, therefore, corresponds to the residual signal which can be considered the (raw—i.e., effects from local lesions, branching points, etc, may present) diffuse plaque component.

In a third example, approximating S3 the healthy lumen comprises applying the spatial domain signal to a component analysis (e.g., PCA or ICA), model trained to determine at least one component that relates healthy lumens to lumens in which diffuse plaque has been introduced. Optionally, local lesions present in the second segment of the vessel show up in a separate component and can be disregarded relatively easy.

PCA, for instance, is a method that reduces the dimensionality of large data sets. PCA reduces the dimensionality of large data sets by transforming a large set of variables into a smaller one that still contains most of the information in the large data set. PCA advantageously reduces the number of variables, hence the simplicity and computational expense of any associated analysis, while minimizing any loss of accuracy.

Performing PCA involves determining the covariance matrix for a set of variables, in this case, variables describing the change in the lumen along the length of the vessel. If the eigenvectors and eigenvalues of the covariance matrix are computed, the percentage contribution to the variance of the data by each of the variables can be determined, meaning that the variables containing the least information can be discarded to leave the principal component(s). The variables that account for the greatest percentage of variation of the data are typically those which provide the most accurate approximation of a healthy lumen (e.g., change in lumen diameter). Therefore, by training the PCA model using information (e.g., angiograms) pertaining to unhealthy lumens, at least one eigenvector can be obtained that corresponds to the variable that accounts for the greatest contribution to the variance of the data (principal component). In this way, one or more eigenvectors are determined that can be used to approximate a healthy lumen given a lumen comprising diffuse plaque. Referring to FIG. 5, the input to the PCA model may be considered information derived from angiograms that corresponds to the line 101 (i.e., a change in a dimension of a lumen of along a length of a vessel), while determination of the eigenvector enables output of information corresponding to line 102 (i.e., approximation of a change in the dimension of a lumen along the length of a healthy vessel). In other words, the signal corresponding to the lumen of an unhealthy vessel in input to the PCA model and the output form the model is a signal corresponding to an approximation of a healthy lumen.

In a fourth example, approximating S3 the healthy vessel comprises applying the spatial domain signal of dimensional variations along the length of the vessel to a neural network trained to determine a spatial domain signal of dimensional variations along the length of a given vessel having a healthy lumen from a spatial domain signal of dimensional variations along the length of the given vessel having diffuse plaque. The neural network may have been trained using spatial domain signals derived from images of healthy lumens and spatial domain signals of lumens in which diffuse plaque and optionally other, significant, local lesions, have been introduced.

In other words, a neural network is trained on synthetically generated or segmented vascular trees, which are modeled by centerline and lumen hull. Into the original lumen hull, diffuse lesions and optionally local lesions are artificially introduced, such that both are known as a ground truth: the healthy and the (diffusely) diseased version of the same vascular tree. Thus, the network can learn to transform a diseased lumen contour into a healthy one, before it is deployed on real cases without ground truth. Accordingly, the neural network is trained to generate a spatial domain signal approximating contours of a healthy vessel burden (e.g., information corresponding to line 102 in FIG. 5) from a spatial domain signal corresponding to contours of a vessel of a patient's lumen that has lesions including a diffuse plaque burden (e.g., information corresponding to line 101 in FIG. 5) and optionally also significant local lesions to be disregarded. Consequently, for a given vessel, the neural network is able to approximate a healthy lumen of the vessel.

Estimating S4 the diffuse plaque burden of the vessel includes subtracting, for the first segment of the vessel, the signal corresponding to the approximation of the healthy lumen from the signal corresponding to the vessel of the lumen (see line 103 in FIG. 5, which shows an estimated diffuse plaque burden). Further, the estimating S4 the diffuse plaque burden of the vessel may include calculating one or more metrics representative of the diffuse plaque burden of the vessel using the signal resulting from the subtraction. In other words, estimating the diffuse plaque burden S4 may include computing a quantity that describes the estimated diffuse plaque burden using the signal resulting from the subtraction. Advantageously, calculating a metric representative of the estimated diffuse plaque burden may engender ease of comparison between the condition of vessels (e.g., between patients) or facilitate tracking of patient history.

In one example, following subtracting the approximation of the healthy lumen from the lumen to obtain an estimated absolute diffuse plaque burden, the estimated absolute diffuse plaque burden may be considered in intervals along the vessel (e.g., intervals of a predetermined width, such as a particular percentage of the total length of the vessel). Thus, by summing the estimated diffuse plaque burden of each interval and by dividing this summation by the number intervals, an estimated mean diffuse plaque burden can be calculated. The number and width of the intervals may be adjustable. From the estimated mean diffuse plaque burden, an estimated variance of the diffuse plaque burden and an estimated standard deviation of the diffuse plaque burden may be straightforwardly calculated.

In another example, a proportion of the vessel having an estimated plaque burden above a predetermined value may be calculated. The predetermined may be a value that has been established as medically significant (e.g., an estimated diffuse plaque burden that is considered to have a deleterious effect on health). For instance, if the estimated absolute diffuse plaque burden is considered in intervals along the vessel, a number of intervals having an estimated diffuse plaque burden above the predetermined value can be calculated. For example, if the estimated absolute diffuse plaque burden is considered for the vessel divided into 100 intervals, it may be that 70 of these 100 intervals have an estimated diffuse plaque burden below the predetermined value and 30 of these intervals have an estimated diffuse plaque burden equal to or above the predetermined value. Therefore, the proportion of the vessel having an estimated diffuse plaque burden above the predetermined value is 30, which may be indicative of the vessel being in poor health.

In another example, the spectral power of the estimated diffuse plaque burden may be calculated by applying a Fourier transformation to the signal corresponding to the estimated diffuse plaque burden and by integrating the signal along the length of the vessel.

The method may comprise outputting the estimation of the diffuse plaque burden to a user interface of, for example, a tablet, smart phone, laptop or alike, meaning that the metric is conveniently viewable by a medical practitioner. The user interface may be configured to display the estimated diffuse plaque burden on top of the vessel in the angiogram received. Similarly, the user interface may be configured to enable highlighting of particular vascular segments (e.g., those with an estimated diffuse plaque burden above a predetermined threshold).

The method may further comprise applying a signal windowing function to the lumen to remove artifacts caused by sudden changes to lumen geometry. A signal windowing function is a function that is zero-valued outside a particular range, typically symmetric about the middle of the range. Sudden changes to lumen geometry (e.g., sudden changes in lumen diameter) may be caused by, for example, localized lesions (as opposed to diffuse lesions/plaque), which generally cause sudden narrowing of the lumen. Sudden changes to lumen geometry may mean that the lumen is not well behaved, which may be associated with branching points. Therefore, advantageously, by applying the signal windowing function, artifacts that may worsen the accuracy of the estimation of the diffuse plaque burden are discarded.

In the same way that the signal windowing function may be applied to the lumen to remove artifacts caused by sudden changes to lumen geometry, the method may further comprise applying a local lesion identifying model configured to identify local lesions (i.e., localized or focal lesions) in the angiogram and removing the local lesions from the angiogram. The local lesion identifying model may be, for example, a neural network trained using paired images of healthy lumens and lumens in which local lesions have been introduced. Advantageously, compared with applying the signal windowing function, applying the local lesion identifying model may more accurately eliminate localized lesions from the estimation of the diffuse plaque burden, resulting in more accurate estimation of the diffuse plaque burden.

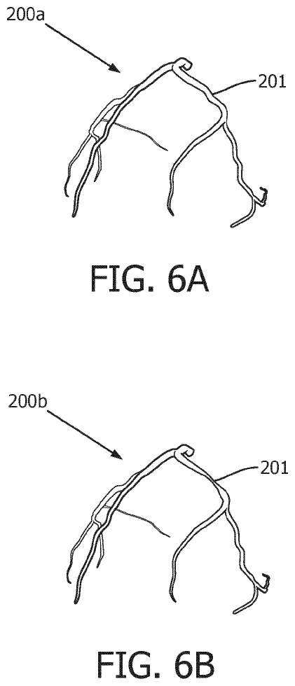

FIGS. 6A and 6B respectively show a vascular tree 200a (without a local lesion) and the same vascular tree 200b in which a local lesion has been introduced. These vascular trees 200a, 200b have been synthetically generated. FIGS. 6A and 6B indicate a position 201 where the local lesion has been synthetically introduced in FIG. 6B.

Similarly, the method may further comprise identifying a branching point in the lumen and assigning a vessel portion including the branching point to the second segment.

A branching point is a point at which a vessel diverges into at least one other vessel. For example, the coronary blood vessels branch into smaller vessels that penetrate the muscle mass to supply blood to the entire heart. Branching points may be identified based on sudden changes in lumen geometry (e.g., diameter) and/or direction, for example. Advantageously, compared with applying the signal windowing function, specifically identifying and removing branching points from the analysis, by assigning them to the second segment being disregarded in the analysis, may more accurately eliminate these parts of the vessel from the estimation of the diffuse plaque burden, resulting in more accurate estimation of the diffuse plaque burden.

In summary, the invention provides a computer-implemented method that enables improved analysis the disease burden of a vessel, in particular the diffuse plaque burden. Consequently, systemic extent and future progression of vascular disease can be better understood.

The optional features set out herein may be used either individually or in combination with each other where appropriate and particularly in the combinations as set out in the accompanying claims. The optional features for each aspect or exemplary embodiment of the invention, as set out herein, are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

All of the features disclosed in this specification (including any accompanying claims and drawings), and/or all of the steps of any method or process so disclosed, may be combined in any combination, except combinations where at most some of such features and/or steps are mutually exclusive.

Although a preferred embodiment has been shown and described, it will be appreciated by those skilled in the art that various changes and modifications might be made without departing from the scope of the invention, as defined in the appended claims and as described above.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measured cannot be used to advantage. Any reference signs in the claims should not be construed as limiting the scope.

1. A computer-implemented method of estimating a diffuse plaque burden of a vessel, the computer-implemented method comprising:
receiving an angiogram including the vessel;
identifying a lumen of the vessel in the angiogram;
applying a local lesion identifying model configured to identify local lesions in the vessel and distinguish a first segment of the vessel from a second segment of the vessel including the identified local lesions;
approximating a healthy lumen of the first segment of the vessel based on determining a change in the lumen along a length of the vessel; and
estimating the diffuse plaque burden of the vessel based on subtracting the approximation of the healthy lumen from the lumen for the first segment of the vessel.

2. The method of claim 1, further comprising:
generating a spatial domain signal of dimensional variations along the length of the vessel at least for the first segment.

3. The method of claim 2, wherein approximating the healthy lumen comprises extracting a low frequency content based on the spatial domain signal using a low-pass filter.

4. The method of claim 2, wherein the approximating the healthy lumen comprises applying the spatial domain signal to an isotonic regression model to fit a line of isotonic regression.

5. The method of claim 2, wherein the approximating the healthy lumen comprises applying the spatial domain signal to a component analysis, CA, model trained to determine an eigenvector that relates healthy lumens to lumens in which diffuse plaque has been introduced.

6. The method of claim 2, wherein approximating the healthy vessel comprises applying the spatial domain signal to a neural network trained to determine a spatial domain signal of a healthy lumen of a vessel from a spatial domain signal of a lumen of the vessel.

7. The method of claim 6, wherein the neural network has been trained using spatial domain signals derived from paired images of healthy lumens and lumens in which diffuse plaque has been introduced.

8. The method of claim 1, wherein identifying the lumen of the vessel comprises, at least for the first segment:
determining a center line of the lumen;
mapping the center line onto a longitudinal axis; and
correcting for any foreshortening of the lumen by resampling the lumen along the longitudinal axis.

9. The method of claim 1, further comprising:
identifying a branching point in the lumen; and
assigning a vessel portion including the branching point to the second segment.

10. The method of claim 1, further comprising:
applying a signal windowing function to the lumen to remove artifacts caused by sudden changes to lumen geometry.

11. The method of claim 1, wherein the estimating the diffuse plaque burden of the vessel is further based on:
calculating a metric representative of the diffuse plaque burden of the vessel, the metric being at least one of: a spectral power; a variance of the estimated plaque burden for intervals along the vessel; a standard deviation of the estimated plaque burden for intervals along the vessel;
an absolute value of the estimated plaque burden; and a proportion of the vessel having an estimated plaque burden above a predetermined value.

12. The method of claim 1, further comprising:
outputting the estimation of the diffuse plaque burden to a user interface.

13. The method of claim 1, wherein the change in the lumen is a change in one of: a radius, a diameter and a cross-sectional area.

14. The method of claim 1, wherein the vessel is a coronary vessel.

15. A transitory, or non-transitory, computer-readable medium having instructions stored thereon which, when executed by a processor, cause the processor to carry out the method of claim 1.
